(19) United States
(12) Patent Application Publication
(10) Pub. No.: US 2025/0221977 A1
(43) Pub. Date: Jul. 10, 2025

(54) NOVEL BENZOTHIOPHENE DERIVATIVE AND USE THEREOF AS BET INHIBITOR

(71) Applicant: BENOBIO CO., LTD., Seongnam-si, Gyeonggi-do (KR)

(72) Inventor: In Hyun LEE, Gwangju (KR)

(73) Assignee: BENOBIO CO., LTD., Seongnam-si, Gyeonggi-do (KR)

(21) Appl. No.: 18/853,963

(22) PCT Filed: Mar. 23, 2023
(86) PCT No.: PCT/KR2023/003859

§ 371 Date: Oct. 3, 2024

(30) Foreign Application Priority Data
Apr. 5, 2022 (KR) 10-2022-0042469

(51) Int. Cl. A61K 31/444; A61K 31/4155; A61K 31/4436; A61K 31/4545; A61P 35/00; C07D 409/04; C07D 409/14

(52) U.S. Cl. CPC A61K 31/444; A61K 31/4155; A61K 31/4436; A61K 31/4545; A61P 35/00; C07D 409/04; C07D 409/14

(57) ABSTRACT

The present invention relates to a novel benzothiophene derivative and use thereof as a bromodomain extra-terminal (BET) inhibitor, and, more specifically, to a novel benzothiophene derivative compound of the following chemical formula 1 having inhibitory activity against a BET protein, and a pharmaceutical composition comprising same for preventing or treating BET protein-associated diseases.

### TECHNICAL FIELD

The present invention relates to a novel benzothiophene derivative and use thereof as a bromodomain extra-terminal (BET) inhibitor, and more particularly, to a novel benzothiophene derivative compound of the following Chemical Formula 1 having inhibitory activity against a BET protein, and a pharmaceutical composition including the benzothiophene derivative compound for preventing or treating BET protein-associated diseases.

### BACKGROUND ART

Post-translational modification (PTM) of histones is involved in the regulation of gene expression and chromatin organization in eukaryotic cells. Histone acetylation at specific lysine residues is a PTM regulated by histone acetylase and histone deacetylase. The histone acetylation controls gene expression by recruiting protein complexes by direct binding of well-conserved proteins called bromodomains to acetylated lysines in histones and other proteins. There are more than 60 bromodomain-containing proteins in the human genome.

Among bromodomain-containing proteins, a Bromodomain Extra-Terminal (BET) family includes BRD2, BRD3, BRD4, and BRDT, and except for BRDT, which is localized in the testis, the remaining proteins are widely expressed in various tissues. In addition, it has been reported that the BET protein family was associated with various diseases, including cancer, metabolic diseases, and inflammation.

For example, oncogenic fusions of BRD4 or BRD3, and nuclear protein in testis (NUT) genes, which are caused by chromosomal translocations, result in aggressive cancer named NUT midline carcinoma (French et al., J Clin Oncol, 22 (2004), 4135-9; French et al., J Clin Pathol, 63 (2008), 492-6). A BRD3/4 bromodomain is conserved in these fusion proteins, and a knockdown or selective BET bromodomain inhibitor, JQ1 causes the death of these cancer cells in both in vitro and an animal tumor model (Filippakopoulos et al., Nature, 468 (2010), 1067-73). It is known that JQ1 and other selective BET inhibitors bind to the BET bromodomain to prevent acetyl-lysine binding, which prevents the BET protein from interacting with chromatin, thereby preventing the transcription from being regulated.

BRD4 was identified as a target in acute myeloid leukemia (AML) by an RNAi screen (Zuber et al., Nature, 478 (2011), 524-8). These findings were validated in vitro and in vivo using the BET inhibitors JQ1 and I-BET151 (Dawson et al., Nature, 478(2011), 529-33). In addition, it is known that the BET inhibitors have broad anticancer activity in acute leukemia, multiple myeloma and other hematologic malignancies. In several cancer models, acute downregulation of the oncogenic transcription factor Myc has been observed upon BET inhibition (Delmore et al., Cell, 146 (2011), 904-17; Mertz et al., Proc Natl Acad Sci US A, 108 (2011), 16669-74). Recent studies suggest that the BET inhibitors have broad potential to be applied to other carcinomas, such as lung cancer and brain cancer.

It has been reported that another BET inhibitor I-BET762, which is closely related to JQ1 in its chemical structure and BET binding mode, regulates the expression of key inflammatory genes in a mouse model and protects the human body from endotoxic shock and bacterial-induced sepsis (Nicodeme et al., Nature, 468 (2010), 1119-23). In addition, these results have been used to support the clinical evaluation of a BET inhibitor RVX-208 in clinical trials in patients with atherosclerosis, coronary artery disease, dyslipidemia, diabetes, and other cardiovascular diseases (McNeill, Curr Opin Investig Drugs, 3 (2010), 357-64 and www.clinicaltrials.gov).

It was found that both RVX-208 and I-BET762 upregulated apolipoprotein A-I, which was important for reducing cholesterol level in the tissues. In addition, it is considered that the BET protein is involved in the proliferation and transcriptional regulation of several viruses, so that the BET inhibitors may have antiviral activity (Weidner-Glunde, Frontiers in Bioscience 15 (2010), 537-549).

Although several bromodomain inhibitors are known clinically and preclinically, there is an urgent need for the development of a new bromodomain inhibitor capable of solving the problems of disease recurrence and resistance to therapeutic agents and reducing side effects.

Meanwhile, redox reactions exist in many physiological processes, and oxygen molecules are required for life, but may produce reactive molecules that lead to a disease. Other reactive chemical species, including free radicals, also cause pathological conditions. It has previously been known that aerobic metabolism related to tissue damage is caused by reactive oxygen species (ROS). The ROS and recently known reactive nitrogen species (RNS), like hormones, are messengers of cell signaling and cause chemical modifications of enzymes and the like and lead to changes in oxidant levels.

In addition, among 20 essential amino acids, cysteine, methionine, tyrosine, and tryptophan are particularly susceptible to oxidation. Accordingly, these proteinaceous substances that are metabolized in the human body cause various modifications, such as metal binding, disulfide bond formation, methylation, acetylation, and the like.

Up to now, studies on the signal regulation mechanisms of cells have been focused on phosphorylation, but the present invention has been completed by applying redox chemistry technology in consideration of the redox control mechanisms such as oxidation, S-nitrosylation, and the like, according to a “redox state” for diseases caused by oxidative and nitrification stress. That is, the present inventors have studied and developed a new bromodomain inhibitor in consideration of the redox state, based on the fact that the degree of binding between signaling substances varies depending on the redox state when the BET inhibitor recognized the lysine residues of the histone protein.

### PRIOR ARTS

### Non-Patent Document

French et al., J Clin Oncol, 22 (2004), 4135-9; French et al., J Clin Pathol, 63 (2008), 492-6

### DISCLOSURE

### Technical Problem

An object of the present invention is to provide a novel benzothiophene derivative compound having excellent inhibitory activity against a BET protein.

Another object of the present invention is to provide a pharmaceutical composition for preventing or treating BET protein-associated diseases, including the compound or pharmaceutically acceptable salt thereof as an active ingredient.

### Technical Solution

Specifically, this will be described as follows. Meanwhile, each description and embodiment disclosed in the present invention can also be applied to each of other descriptions and embodiments. That is, all combinations of various components disclosed in the present invention belong to the scope of the present invention. In addition, the specific description described below may not limit the scope of the present invention.

The present invention provides a benzothiophene derivative compound represented by Chemical Formula 1 below or a pharmaceutically acceptable salt thereof.

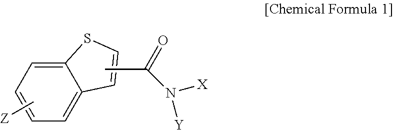

Wherein,
X or Y may be each independently H, halo, cyano, alkyl, alkenyl, alkynyl, —(Rₐ)C(═O)R_{b}, —(Rₐ)C(═O)N(R_{b})(R_{c}), —(Rₐ)C(═O)OR_{b}, cycloalkyl, heterocycloalkyl, aryl, heteroaryl, pyridine, alkyl pyridine, piperidine, piperidinopyridine, indazole, or

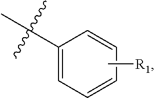

Z may be the same or different one or more selected from the group consisting of H, C₁-C₆ alkyl, —ORₐ, —SRₐ, cycloalkyl, heterocycloalkyl, aryl, heteroaryl, pyridine, pyrazole, alkylpyrazole, and hydroxypyridine,
Rₐ, R_{b} and R_{c} may be each independently H, alkyl, or alkylene, and
R₁ may be H, halo, cyano, alkyl, alkenyl, alkynyl, or halogenated methyl.

According to another aspect of the present invention, there is provided a pharmaceutical composition for preventing or treating BET protein-associated diseases, including the compound or pharmaceutically acceptable salt thereof.

The BET protein-associated disease may be at least one selected from the group consisting of cancer; autoimmune or inflammatory diseases; metabolic diseases; and viral diseases.

### Advantageous Effects

The benzothiophene derivative compound represented by Chemical Formula 1 provided in the present invention has excellent inhibitory activity against BET proteins, and thus can be usefully used as an agent for preventing or treating various diseases related to BET.

### DESCRIPTION OF DRAWINGS

FIGS. 1 to 14 illustrate structural information of synthesized compounds 1 to 7.

FIG. 15 illustrates results of an in vitro anticancer efficacy test on an AML cell line.

FIG. 16 illustrates results of an in vivo anticancer efficacy test on human pancreatic cancer cells.

### MODES OF THE INVENTION

Hereinafter, various embodiments of the present invention will be described in detail with reference to the accompanying drawings. The present invention is not limited to specific embodiments, and it should be understood to include various modifications, equivalents, and/or alternatives to the embodiments of the present invention. In connection with the description of the drawings, similar reference numerals may be used for similar components.

In this specification, expressions such as “have,” “may have,” “include,” or “may include” refer to the presence of the corresponding feature (e.g., numerical value, function, operation, or component such as part), and does not exclude the presence of additional features.

In the present invention, the expression such as “A or B”, “at least one of A and/or B”, or “one or more of A and/or B” may include all possible combinations of items listed together. For example, “A or B”, “at least one of A and B”, or “at least one of A or B” may refer to all cases of (1) including at least one A, (2) including at least one B, or (3) including both at least one A and at least one B.

The expression of “configured to” used herein may be changed and used to, for example, “suitable for”, “having the capacity to”, “designed to”, “adapted to”, “made to” or “capable of”, depending on the situation. The term of “configured to” may not necessarily mean “specially designed to” in hardware.

The terms used herein are used to illustrate only specific exemplary embodiments, and may not be intended to limit the scope of other exemplary embodiments. A singular expression may include a plural expression unless the context clearly dictates otherwise. The terms used herein, including technical or scientific terms, may have the same meaning as generally understood by those of ordinary skill in the art described in the present invention. The terms defined in a general dictionary among the terms used herein may be interpreted in the same or similar meaning as or to the meaning on the context of the related art, and will not be interpreted as an ideal or excessively formal meaning unless otherwise defined in the present invention. In some cases, even the terms defined in the present invention cannot be interpreted to exclude the exemplary embodiments of the present invention.

The exemplary embodiments disclosed in the present invention are presented for explanation and understanding of the disclosed technical contents, and do not limit the scope of the present invention. Therefore, the scope of the present invention should be interpreted as including all changes or various other embodiments based on the technical idea of the present invention.

Hereinafter, a preferred exemplary embodiment of the present invention will be described in detail. Terms and words used in the present specification and claims should not be interpreted as being limited to typical or dictionary meanings, but should be interpreted as having meanings and concepts which comply with the technical spirit of the present invention, based on the principle that an inventor can appropriately define the concept of the term to describe his/her own invention in the best manner.

The configurations in the embodiments described in the present specification are merely the most preferred embodiment of the present invention and are not intended to represent all of the technical ideas of the present invention, and thus, it should be understood that there may be various equivalents and modifications capable of replacing the embodiments at the time of this application.

Throughout the specification, when a part “includes” a component, unless otherwise specifically stated, it is meant to further include other components rather than excluding other components.

Hereinafter, the present invention will be described in detail.

The present invention relates to a novel benzothiophene derivative compound, and more particularly, to a novel benzothiophene derivative compound having inhibitory activity against a BET protein, and a pharmaceutical composition including the novel benzothiophene derivative compound for preventing or treating BET protein-associated diseases.

Unless otherwise stated, terms used in the description and claims of the present invention have the meanings set forth below.

According to the convention used in the art, in Chemical Formula herein,

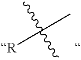

is used to indicate that a moiety or substituent “R” is attached to a skeleton structure.

“Alkyl” is hydrocarbon having primary, secondary, tertiary and/or quaternary carbon atoms and includes a saturated aliphatic group which may be straight-chain, branched or cyclic, or a combination thereof. For example, the alkyl group may have 1 to 20 carbon atoms (i.e., C₁-C₂₀ alkyl), 1 to 10 carbon atoms (i.e., C₁-C₁₀ alkyl), or 1 to 6 carbon atoms (i.e., C₁-C₆ alkyl). Unless otherwise defined, in a preferred embodiment, the alkyl refers to C₁-C₆ alkyl. Examples of suitable alkyl groups may include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)₇CH₃), but are not limited thereto.

Moreover, as used throughout the specification, examples and claims, the term “alkyl” is intended to include both unsubstituted and substituted alkyl groups, the latter thereof refers to an alkyl moiety having a substituent that replaces hydrogen on one or more carbons of the hydrocarbon backbone, which includes a haloalkyl group such as trifluoromethyl, 2,2,2-trifluoroethyl, and the like.

When used with a chemical moiety such as acyl, acyloxy, alkyl, alkenyl, alkynyl or alkoxy, the term “C_{x-y}” or “Cₓ—C_{y}” is intended to include a group containing x to y carbons in the chain. C₀ alkyl represents hydrogen when the group is located in the terminal position or a bond when the group is located inside. For example, a (C₁-C₆)alkyl group contains 1 to 6 carbon atoms in the chain.

“Alkoxy” refers to a group with Chemical Formula —O-alkyl, in which the alkyl group as defined above is attached to a parent compound through oxygen atoms. The alkyl moiety of the alkoxy group may have, for example, 1 to 20 carbon atoms (i.e., C₁-C₂₀ alkoxy), 1 to 12 carbon atoms (i.e., C₁-C₁₂ alkoxy), 1 to 10 carbon atoms (i.e., C₁-C₁₀ alkoxy), or 1 to 6 carbon atoms (i.e., C₁-C₆ alkoxy). Examples of suitable alkoxy groups may include methoxy (—O—CH₃ or —OMe), ethoxy (—OCH₂CH₃ or —OEt), and t-butoxy (—OC(CH₃)₃ or —O-tBu), but are not limited thereto.

“Alkenyl” is a hydrocarbon that has primary, secondary, tertiary and/or quaternary carbon atoms, includes straight-chain, branched and cyclic groups, or a combination thereof, and has at least one unsaturated region, that is, a carbon-carbon sp² double bond. For example, the alkenyl group may have 2 to 20 carbon atoms (i.e., C₂-C₂₀ alkenyl), 2 to 12 carbon atoms (i.e., C₂-C₁₂ alkenyl), 2 to 10 carbon atoms (i.e., C₂-C₁₀ alkenyl), or 2 to 6 carbon atoms (i.e., C₂-C₆ alkenyl). Examples of suitable alkenyl groups may include vinyl (—CH═CH₂), allyl (—CH₂CH═CH₂), cyclopentenyl (—C₅H₇), and 5-hexenyl (—CH₂CH₂CH₂CH₂CH═CH₂), but are not limited thereto.

“Alkynyl” is a hydrocarbon that has primary, secondary, tertiary and/or quaternary carbon atoms, includes straight-chain, branched and cyclic groups, or a combination thereof, and has at least one carbon-carbon sp triple bond. For example, the alkynyl group may have 2 to 20 carbon atoms (i.e., C₂-C₂₀ alkynyl), 2 to 12 carbon atoms (i.e., C₂-C₁₂ alkynyl), 2 to 10 carbon atoms (i.e., C₂-C₁₀ alkynyl), or 2 to 6 carbon atoms (i.e., C₂-C₆ alkynyl). Examples of suitable alkynyl groups may include acetylenic (—C≡CH) and propargyl (—CH₂C≡CH), but are not limited thereto.

As used herein, the term “aryl” includes a substituted or unsubstituted monovalent or divalent aromatic hydrocarbon group, which is monocyclic, bicyclic or polycyclic where each atom of the ring is carbon. Preferably, an aryl ring is a 6- to 20-membered ring, a 6- to 14-membered ring, a 6- to 10-membered ring, or more preferably a 6-membered ring. The aryl group may be a polycyclic ring system having two or more cyclic rings in which two or more carbons are common to two adjacent rings, wherein one or more of the rings may be aromatic, and for example, the other cyclic rings may be cycloalkyl, cycloalkenyl, cycloalkynyl, aryl, heteroaryl, and/or heterocycloalkyl. The aryl group may include benzene, naphthalene, phenanthrene, anthracene, indene, indane, phenol, aniline, and the like.

As used herein, the term “carbocyclylalkyl”, “cycloalkylalkyl”, or “(cycloalkyl)alkyl” refers to an alkyl group substituted with a carbocycle group or a cycloalkyl group.

As used herein, the term “carbocycle”, “carbocyclyl”, “carbocyclic”, or “cycloalkyl” may be monocyclic, bicyclic, or polycyclic and refers to a non-aromatic saturated or unsaturated, monovalent or divalent ring where each ring atom is carbon. The cycloalkyl group may have 3 to 7 carbon atoms as a monocycle, 7 to 12 carbon atoms as a bicycle, and about 20 carbon atoms or less as a polycycle. Monocyclic cycloalkyl has 3 to 7 ring atoms, more typically 5 or 6 ring atoms. Bicyclic cycloalkyl may have 7 to 12 ring atoms, and may be a fused ring system, a spirocyclic ring system, or a bridged ring system. In an exemplary cycloalkyl group, the atoms may be arranged in a bicyclo[4,5], [5,5], [5,6], or [6,6] system. In a specific embodiment, cycloalkyl contains 3 to 20 atoms, or 3 to 10 atoms, or more preferably 3 to 7 atoms. Examples of cycloalkyls may include cyclopropyl, cyclobutyl, cyclopentyl, cyclohexyl, cycloheptyl, cyclooctyl, and the like. Unless otherwise specified, cycloalkyl may be substituted by one or more substituents described herein.

As used herein, the terms “heterocyclylalkyl” and “heterocycloalkyl” refer to an alkyl group substituted with a heterocycloalkyl group.

The terms “heterocyclyl”, “heterocycle”, “heterocyclic”, and “heterocycloalkyl” refer to substituted or unsubstituted, monovalent or divalent, saturated or partially saturated non-aromatic ring structures, preferably 3- to 10-membered rings, more preferably 3- to 7-membered rings, in which the ring structure contains at least 1 heteroatom, preferably 1 to 4 heteroatoms, and more preferably 1 to 2 heteroatoms. The terms “heterocyclyl”, “heterocycle”, “heterocyclic”, and “heterocycloalkyl” may also include a polycyclic ring system having two or more cyclic rings where two or more carbons are common to two adjacent rings, wherein one or more of the rings may be heterocyclic, and for example, the other cyclic ring may be cycloalkyl, cycloalkenyl, cycloalkynyl, aryl, heteroaryl, and/or heterocycloalkyl. Bicyclic and polycyclic heterocyclic ring systems may be fused, bridged, or spiro ring systems. Substituted heterocycle includes, for example, a heterocyclic ring substituted with any substituent disclosed herein, including a carbonyl group. The heterocyclyl group includes, for example, piperidine, piperazine, pyrrolidine, morpholine, lactone, lactam, and the like. Additional exemplary heterocyclos may include dihydropyridyl, dihydroindolyl, tetrahydropyridyl (piperidyl), tetrahydrothiophenyl, sulfur-oxidized tetrahydrothiophenyl, indolenyl, piperidinyl, 4-piperidinyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, 6H-1,2,5-thiadiazinyl, 2H,6H-1,5,2-dthiazinyl, pyranyl, chromenyl, xanthenyl, phenoxatinyl, 2H-pyrrolyl, 3H-indolyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, quinuclidinyl, morpholinyl, and oxazolidinyl (each of these may be substituted or unsubstituted), but are not limited thereto.

“Heteroaryl” refers to a substituted or unsubstituted monovalent or divalent aromatic group containing one or more heteroatoms in the ring, which is monocyclic, bicyclic or polycyclic. Non-limiting examples of a suitable heteroatom that may be contained in the aromatic ring may include oxygen, sulfur, and nitrogen. In a polycyclic heteroaryl ring system, the ring system has two or more cyclic rings where two or more carbons are common to two adjacent rings, wherein one or more of the rings may be heteroaromatic, and for example, other cyclic rings may be cycloalkyl, cycloalkenyl, cycloalkynyl, aryl, heteroaryl, and/or heterocycloalkyl.

The hetero group includes, for example, benzofuran, benzothiophene, pyrrole, furan, thiophene, imidazole, indole, isoindole, isoxazole, isothiazole, oxazole, thiazole, quinoline, isoquinoline, pyrazole, pyridine, pyrazine, pyridazine, pyrimidine, etc. (each of these may be substituted or unsubstituted).

As used herein, the terms “halo” and “halogen” refer to halogen and include chloro, fluoro, bromo, and iodo.

“Amino” refers to a —NH₂ group.

“Cyano” refers to a —CN group.

“Nitro” refers to a —NO₂ group.

“Carboxy” refers to a —C(O)OH group.

“Aldehyde” refers to a —CHO group.

“Alkoxycarbonyl” refers to a —C(O)O(alkyl) or —C(O)O(cycloalkyl) group, wherein the alkyl and cycloalkyl are as defined above.

“Acyl halide” refers to a compound including a —C(O)-halogen group.

The present invention provides a benzothiophene derivative compound represented by Chemical Formula 1 below or a pharmaceutically acceptable salt thereof.

### [Chemical Formula 1]

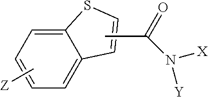

wherein,
X or Y is each independently H, halo, cyano, alkyl, alkenyl, alkynyl, —(Rₐ)C(═O)R_{b}, —(Rₐ)C(═O)N(R_{b})(R_{c}), —(Rₐ)C(═O)OR_{b}, cycloalkyl, heterocycloalkyl, aryl, heteroaryl, pyridine, alkyl pyridine, piperidine, piperidinopyridine, indazole, or

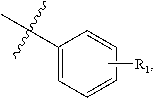

Z may be the same or different one or more selected from the group consisting of H, C₁-C₆ alkyl, —ORₐ, —SRₐ, cycloalkyl, heterocycloalkyl, aryl, heteroaryl, pyridine, pyrazole, alkylpyrazole, and hydroxypyridine,
Rₐ, R_{b} and R_{c} are each independently H, alkyl, or alkylene, and
R₁ is H, halo, cyano, alkyl, alkenyl, alkynyl, or halogenated methyl.

In one embodiment, X may be —(Rₐ)C(═O)R_{b} or —(Rₐ)C(═O)N(R_{b})(R_{c}), Y is

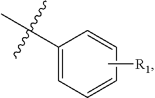

Z may be the same or different one or more selected from the group consisting of —ORₐ and hydroxypyridine, and R¹ may be H, halo, cyano, alkyl, alkenyl, alkynyl, or halogenated alkyl.

In one embodiment, X may be —(Rₐ)C(═O)N(R_{b})(R_{c}), and R₁ may be fluoroalkyl, chloroalkyl, or bromoalkyl as halogenated alkyl.

In one embodiment, Z may be the same or different one or more selected from the group consisting of —ORₐ and

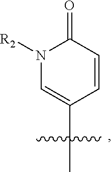

and R₂ may be H or alkyl.

Preferred examples of the compound of Chemical Formula 1 according to the present invention are as shown in Table 1 below, but are not limited thereto.

TABLE 1

| No | Compound name | Chemical structure |
|---|---|---|
| 2 | BBC1115 | 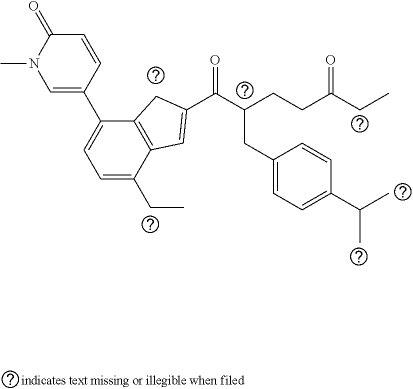 [SMILES: CCC(=O)CCC(Cc1ccc(C(C)C)cc1)C(=O)C1=Cc2c(CC)ccc(-c3ccc(=O)n(C)c3)c2C1] |
| 3 | BBC1114 | 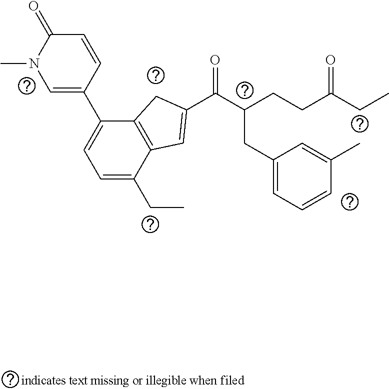 [SMILES: CCC(=O)CCC(Cc1cccc(C)c1)C(=O)C1=Cc2c(CC)ccc(-c3ccc(=O)n(C)c3)c2C1] |
| 4 | BBC1117 | 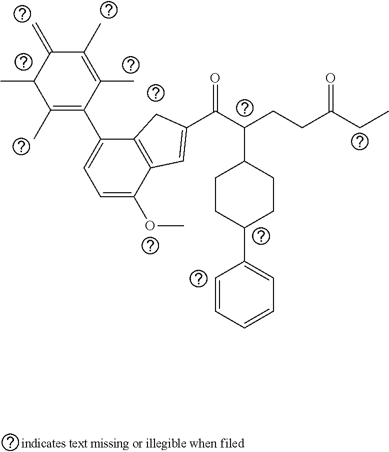 [SMILES: C=C1C(C)=C(C)C(c2ccc(OC)c3c2CC(C(=O)C(CCC(=O)CC)C2CCC(c4ccccc4)CC2)=C3)=C(C)C1C] |
| 5 | BBC1118 | 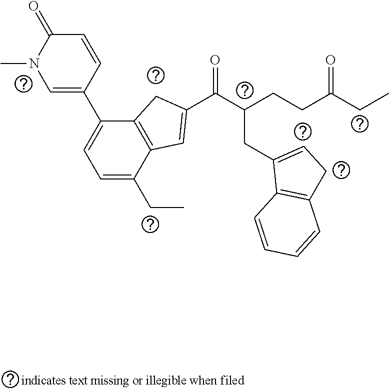 [SMILES: CCC(=O)CCC(CC1=CCc2ccccc21)C(=O)C1=Cc2c(CC)ccc(-c3ccc(=O)n(C)c3)c2C1] |
| 6 | BBC1131 | 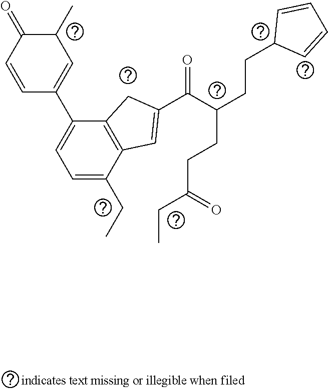 [SMILES: CCC(=O)CCC(CCC1C=CC=C1)C(=O)C1=Cc2c(CC)ccc(C3=CC(C)C(=O)C=C3)c2C1] |
| 7 | BBC1674 | 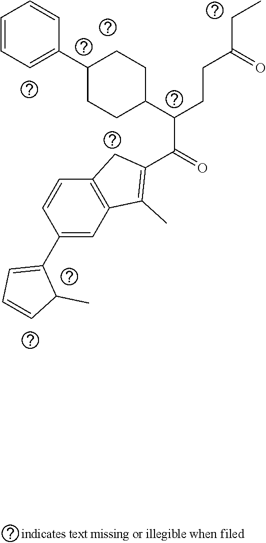 [SMILES: CCC(=O)CCC(C(=O)C1=C(C)c2cc(C3=CC=CC3C)ccc2C1)C1CCC(c2ccccc2)CC1] |
| 8 | BBC1680 | 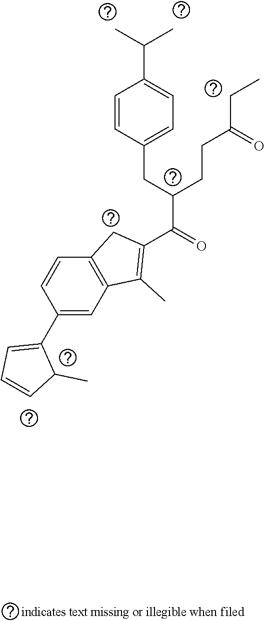 [SMILES: CCC(=O)CCC(Cc1ccc(C(C)C)cc1)C(=O)C1=C(C)c2cc(C3=CC=CC3C)ccc2C1] |

The compound according to the present invention may form a pharmaceutically acceptable salt. The pharmaceutically acceptable salt is not particularly limited as long as it is an acid that forms a non-toxic acid addition salt containing a pharmaceutically acceptable anion. For example, the pharmaceutically acceptable salt may include acid addition salts formed by inorganic acids such as hydrochloric acid, sulfuric acid, nitric acid, phosphoric acid, hydrobromic acid, hydroiodic acid, and the like; organic acids such as tartaric acid, formic acid, citric acid, acetic acid, trichloroacetic acid, trifluoroacetic acid, gluconic acid, benzoic acid, lactic acid, fumaric acid, maleic acid, and the like; and sulfonic acids such as methanesulfonic acid, benzenesulfonic acid, p-toluenesulfonic acid, naphthalenesulfonic acid, and the like.

In another aspect, the present invention provides a pharmaceutical composition for preventing or treating bromodomain extra-terminal (BET) protein-associated diseases, containing the compound represented by Chemical Formula 1 or pharmaceutically acceptable salt thereof as an active ingredient.

The pharmaceutical composition of the present invention is useful for preventing or treating various diseases associated to the BET protein because the compound represented by Chemical Formula 1 contained therein inhibits the BET proteins. The BET protein-associated diseases may be cancer; autoimmune or inflammatory diseases; metabolic diseases; or viral diseases.

In one embodiment of the present invention, the cancer may be at least one selected from the group consisting of hematologic cancer, multiple myeloma, acute myeloid leukemia, malignant lymphoma, aplastic anemia, thymus cancer, ovarian cancer, cervical cancer, breast cancer, colorectal cancer, liver cancer, stomach cancer, pancreatic cancer, colon cancer, peritoneal metastatic cancer, skin cancer, bladder cancer, prostate cancer, thyroid cancer, lung cancer, osteosarcoma, fibrous tumor, and brain tumor, but is not limited thereto.

In one embodiment of the present invention, the autoimmune or inflammatory disease may be at least one selected from the group consisting of rheumatoid arthritis, systemic lupus erythematosus, multiple sclerosis, Type 1 diabetes, hyperthyroidism, myasthenia, Crohn's disease, ankylosing spondylitis, psoriasis, autoimmune pernicious anemia and Sjogren's syndrome, allergy, allergic rhinitis, arthritis, asthma, chronic obstructive pulmonary disease, degenerative joint disease, dermatitis, organ rejection, eczema, hepatitis, inflammatory bowel disease, sepsis, sepsis syndrome, septic shock, and nonalcoholic steatohepatitis, but is not limited thereto.

In one embodiment of the present invention, the metabolic disease may be at least one selected from the group consisting of hypertriglyceridemia, obesity, hyperlipidemia, hyperinsulinemia, hyperglycemia, arteriosclerosis, hypertension, Type 2 diabetes, and insulin resistance disease, but is not limited thereto.

In one embodiment of the present invention, the viral disease may be at least one selected from the group consisting of infantile paralysis, acute hemorrhagic conjunctivitis, viral meningitis, hand, foot and mouth disease, hepatitis, myositis, myocarditis, pancreatitis, epidemic myalgia, encephalitis, common cold, herpangina, foot and mouth disease, asthma, bronchiolitis, bronchitis, chronic obstructive pulmonary disease, pneumonia, sinusitis, otitis media, herpes simplex, herpes zoster, stomatitis, and chickenpox, but is not limited thereto.

According to another embodiment of the present invention, there is provided a pharmaceutical formulation including the pharmaceutical composition.

The pharmaceutical formulation of the present invention may be in various oral dosage forms such as tablets, pills, powders, capsules, syrups or emulsions, or parenteral dosage forms such as injections, for example, injections for intramuscular, intravenous or subcutaneous administration, and preferably oral dosage forms.

In addition, the pharmaceutical formulation may be formulated according to a conventional method by adding conventional non-toxic pharmaceutically acceptable additives, which are one or more selected from the group consisting of carriers, additives, and excipients as a specific example, in addition to the active ingredient.

Hereinafter, the present invention will be described in more detail by the following Examples. However, these Examples are only illustrative of the present invention, and the scope of the present invention is not limited to these Examples.

### Example 1: Synthesis of Compound 1 (BBC1115)

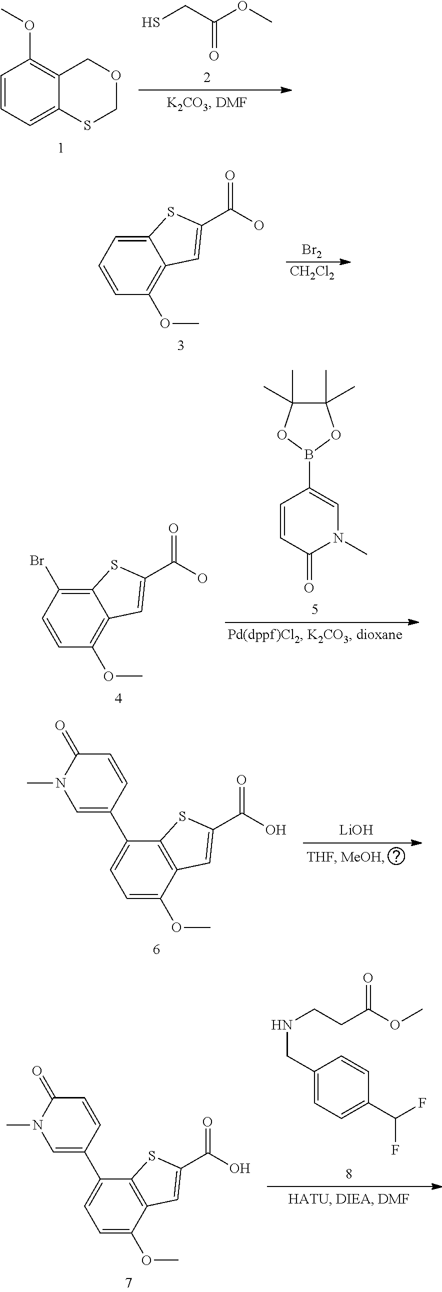

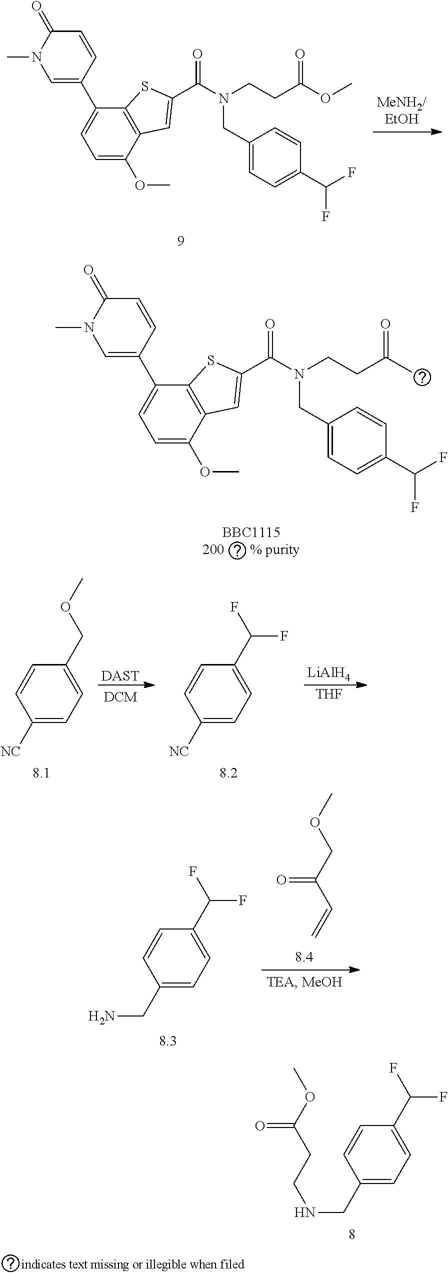

From tert-butyl 4-formylbenzylcarbamate (200 mg, 850 μmol) in DCM (3 mL), N-[[4-(difluoromethyl)phenyl]methyl]-4-methoxy-N-[3-(methylamino)-3-oxo-propyl]-7-(1-methyl-6-oxo-3-pyridyl)benzothiophene-2-carboxamide (BBC1115) (20.9 mg, 37.8 μmol, 20.5% yield, 97.7% purity) was obtained by Reaction Scheme above.

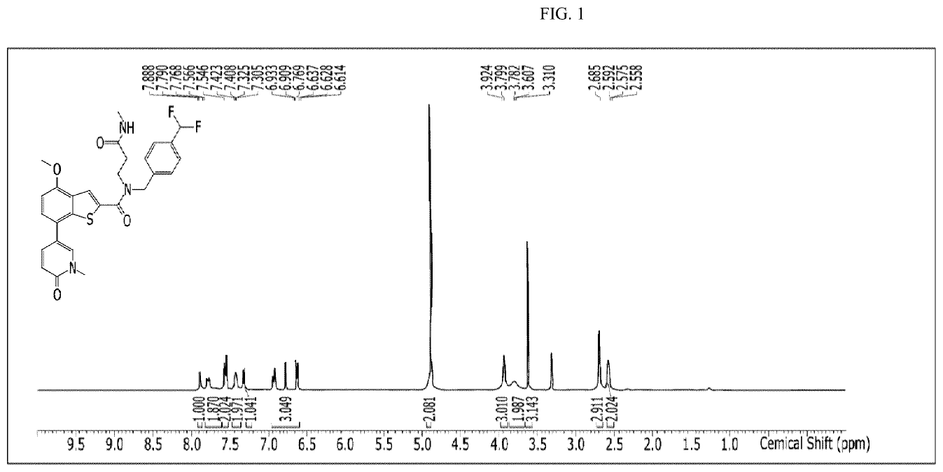

1H NMR: EB5637-22-P1H1 (400 MHz, CD3OD) δ 7.88 (s, 1H), 7.76-7.90 (m, 2H), 7.56 (d, J=8.0 Hz, 2H), 7.40-7.44 (m, 2H), 7.32 (d, J=8.0 Hz, 2H), 6.61-6.93 (m, 3H), 4.80-4.94 (m, 2H), 3.92 (s, 3H), 3.78-3.79 (m, 2H), 3.60 (s, 3H), 2.68 (s, 3H), 2.57 (t, J=6.8 Hz, 2H) (FIG. 1)

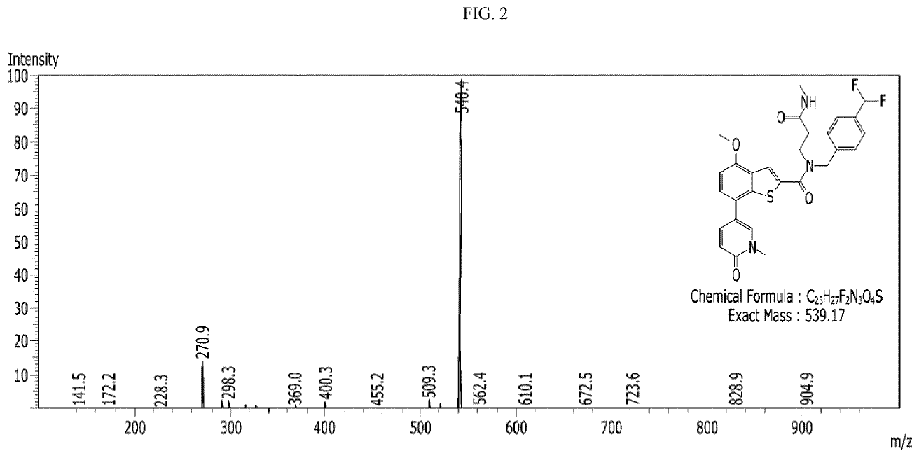

LCMS: EB5637-22-P1L1, m/z=540.4 (M+H)+, Rt=1.685 min (FIG. 2)

### Example 2: Synthesis of Compound 2 (BBC1114)

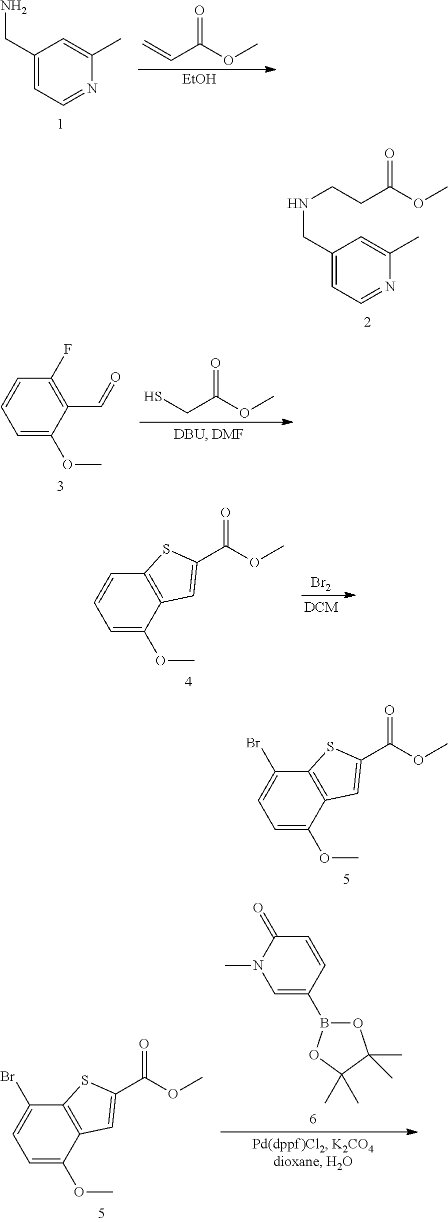

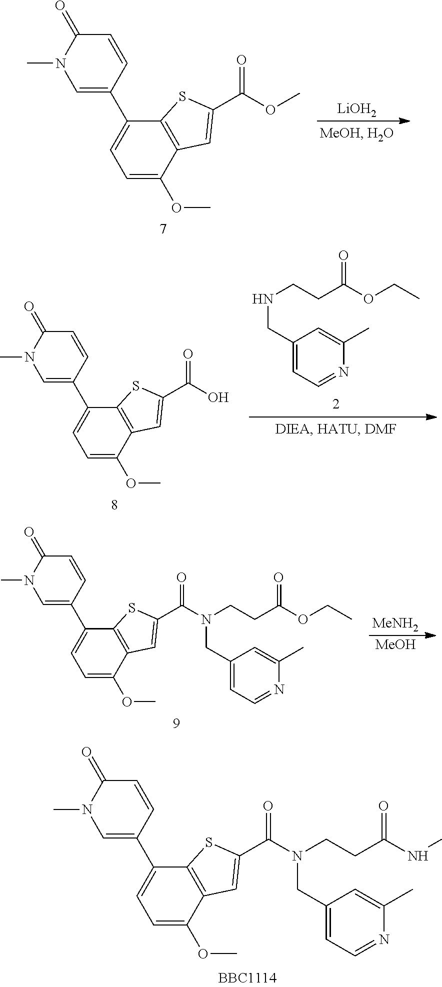

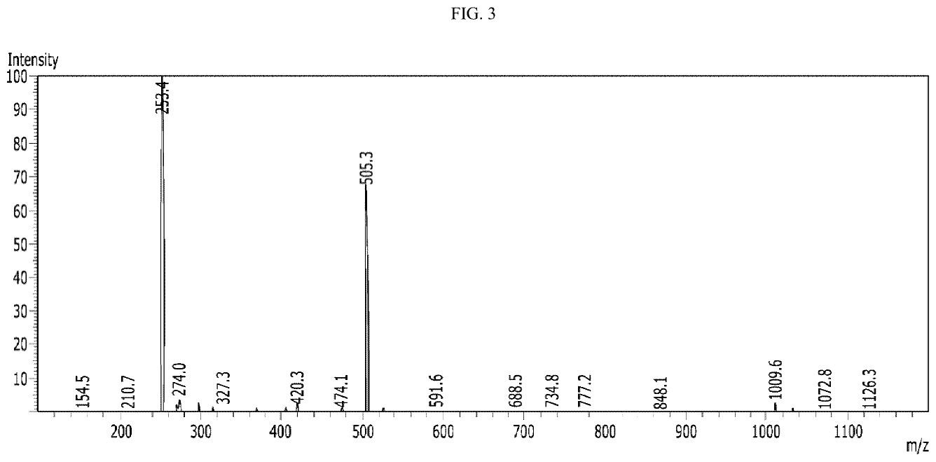

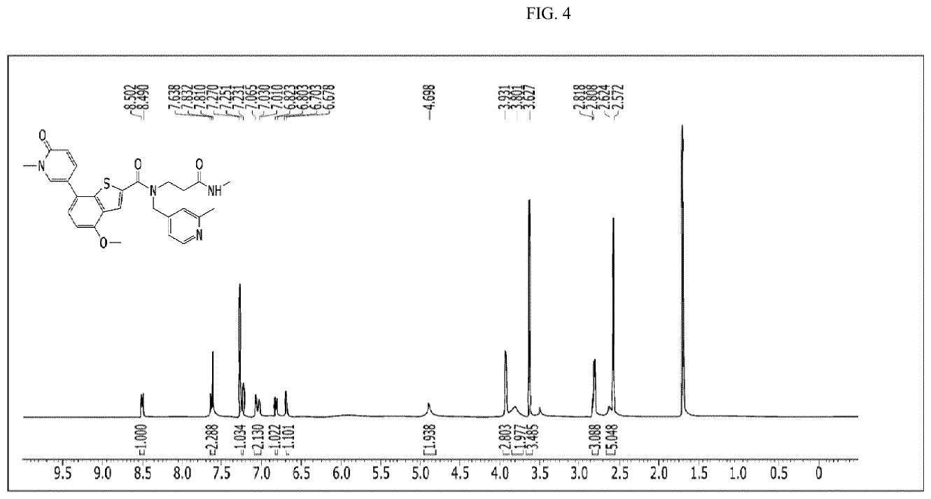

As shown in the synthetic pathway, 4-methoxy-N-[13-(methylamino)-3-oxo propyl]-7-(1-methyl-6-oxo-3-pyridyl)-N-[(2-methyl-4-pyridyl)methyl]benzothiophene-2-carboxamide (BBC1 114) (21.6 mg, 42.6 μmol, 14.8% yield, 99.5% purity) was obtained from (2 methylpyridin 4 yl) methanamine (100 mg, 825 μmol). The structure was confirmed by H-NMR and the purity was confirmed by LCMS (FIGS. 3 and 4).

H NMR: EB1433-145-P1N2, (400 MHz, CDCl3)

δ8.5-8.5 (m, 1H), 7.6-7.7 (m, 3H), 7.2-7.3 (m, 1H), 7.0-7.1 (m, 2H), 6.8-6.9 (m, 1H), 6.7-6.8 (m, 1H), 4.8-5.0 (m, 2H), 3.9-4.0 (m, 3H), 3.7-3.9 (m, 2H), 3.6-3.7 (m, 3H), 2.8-2.9 (m, 3H), 2.5-2.7 (m, 5H)

LCMS: EB1433 145 P1Z (M+H)+: 505.3

### Example 3: Synthesis of Compound 3 (BBC1117)

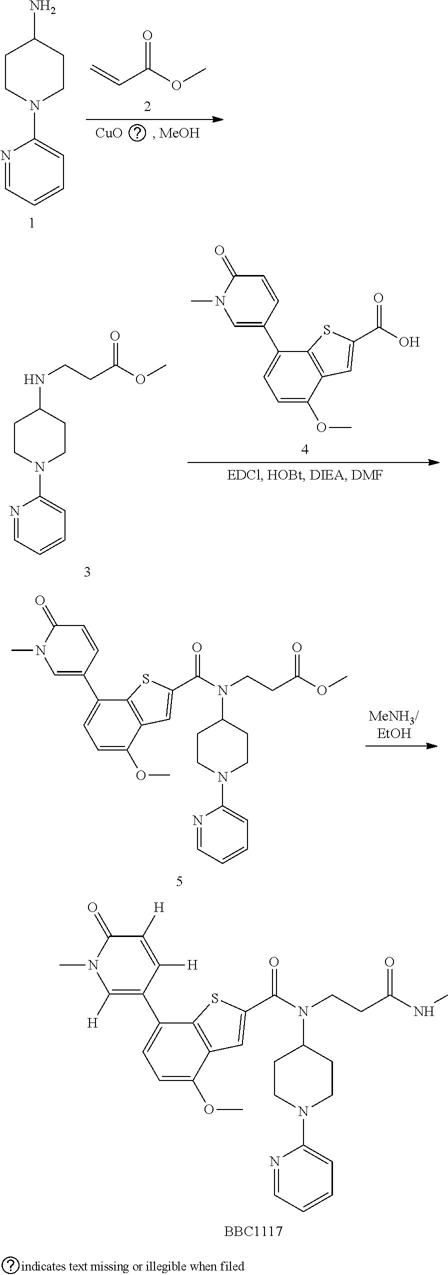

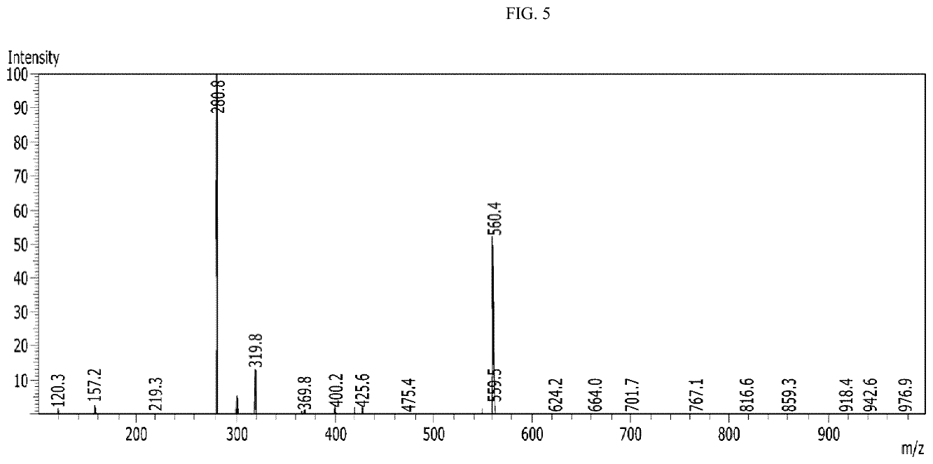

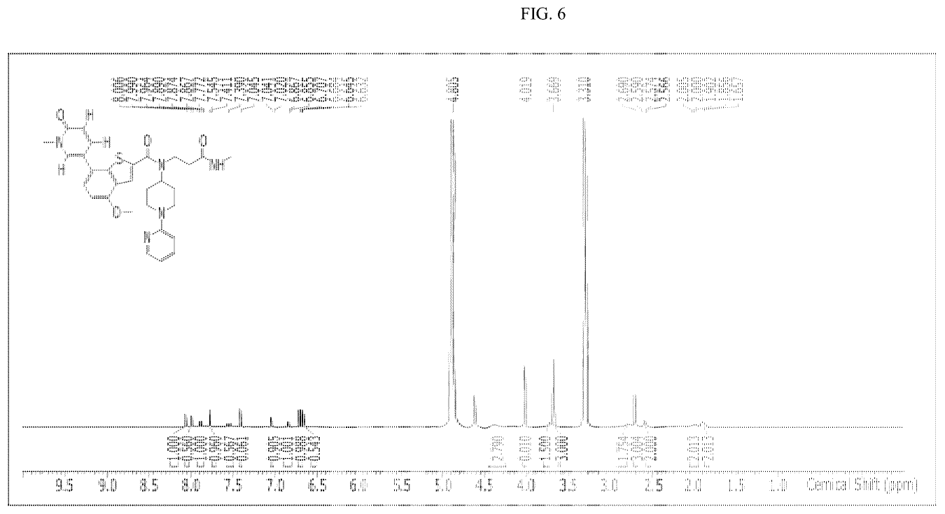

As shown in the synthetic pathway, 4-methoxy-7-(1-methyl-6-oxo-1, 6-dihydropyridin-3-yl)-N-(3-(methylamino)-3-oxopropyl)-N-(1-(pyridine-2-yl)piperidin-4-yl)benzo[b]thiophene-2-carboxamide (BBC1117) (11.7 mg, 20.0 μmol, 28.0% yield, 96.0% purity) was obtained from 1-(pyridine-2-yl)piperidin-4-amine (165 mg, 930 μmol). The structure was confirmed by H-NMR and the purity was confirmed by LCMS (FIGS. 5 and 6).

H NMR: EB2096-65-P1A1, (400 MHz, MeOD)

δ8.07 (s, 1H), 7.99 (d, J=2.4 Hz, 1H), 7.88 (dd, J=2.4, 1H), 7.77 (s, 1H), 7.58-7.50 (m, 1H), 7.40 (d, J=8.0, 1H), 7.03 (d, J=8, 1H), 6.85 (d, J=8.8, 1H), 6.70 (d, J=9.2, 1H), 6.65 (dd, J=4.8, 1H), 4.61 (s, 4H), 4.38 (br d, J=12.8, 2H), 4.02 (s, 3H), 3.71 (br s, 1H), 3.67 (s, 3H), 2.71-2.72 (m, 1H), 2.70 (s, 2H), 2.55-2.61 (m, 2H), 1.93-2.05 (m, 2H), 1.83-1.90 (m, 2H)

LCMS: EB1745-65-P1A1, [M+H]+, 560.4

### Example 4: Synthesis of Compound 4 (BBC1118)

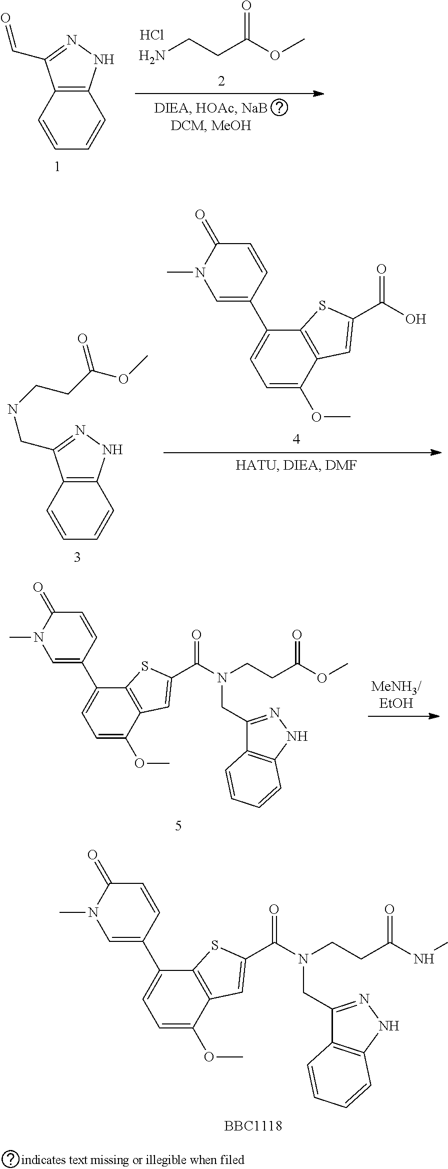

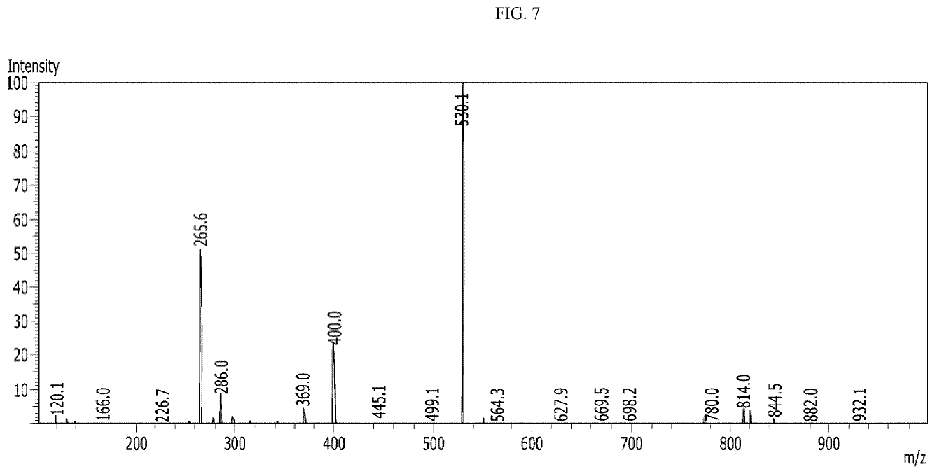

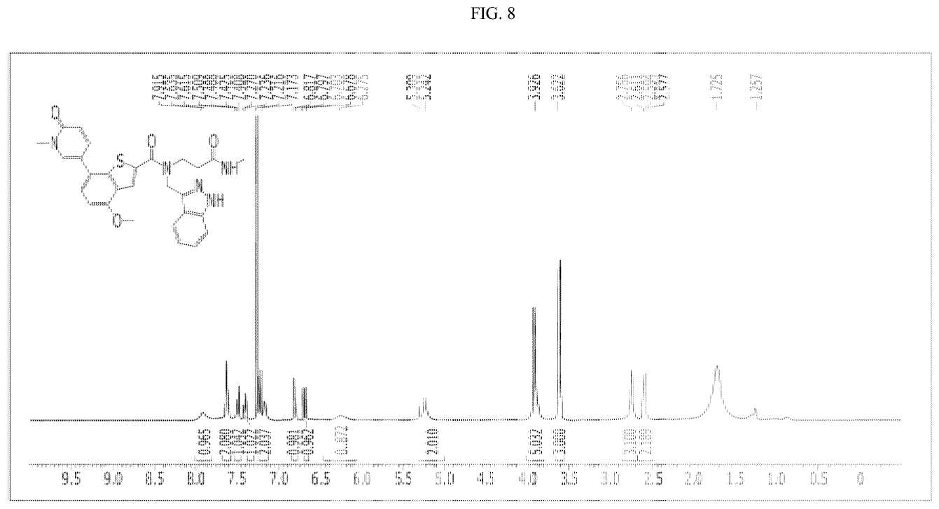

As shown in the synthetic pathway, N-((1H-indazol-3-yl)methyl)-4-methoxy-7-(1-methyl-6-oxo-1,6-dihydropyridin-3-yl)-N-(3-(methylamino)-3-oxopropyl)benzo[b]thiophene-2-carboxamide (BBC11 18) (6.30 mg, 11.9 μmol, 11.4% yield, 99.7% purity) was obtained from 1H-indazole-3-carbaldehyde (500 mg, 3.42 mmol). The structure was confirmed by HNMR and the purity was confirmed by L CMS (FIGS. 7 and 8).

LCMS: (EB1359-132-P1A1), (M+1)+, 529.1

H NMR: EB1359-132-P1A1 (400 MHz, CDCl3)

### Example 5: Synthesis of Compound 5 (BBC1131)

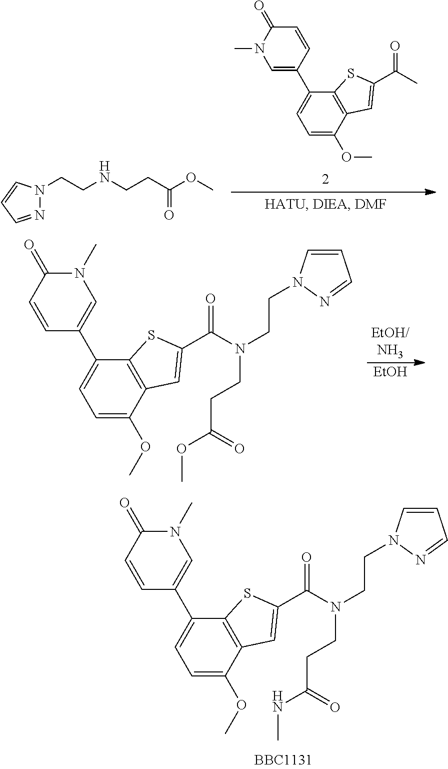

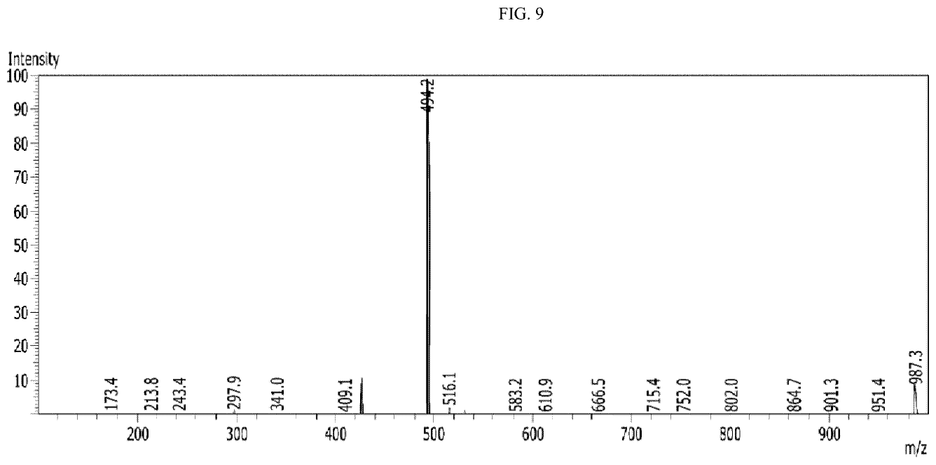

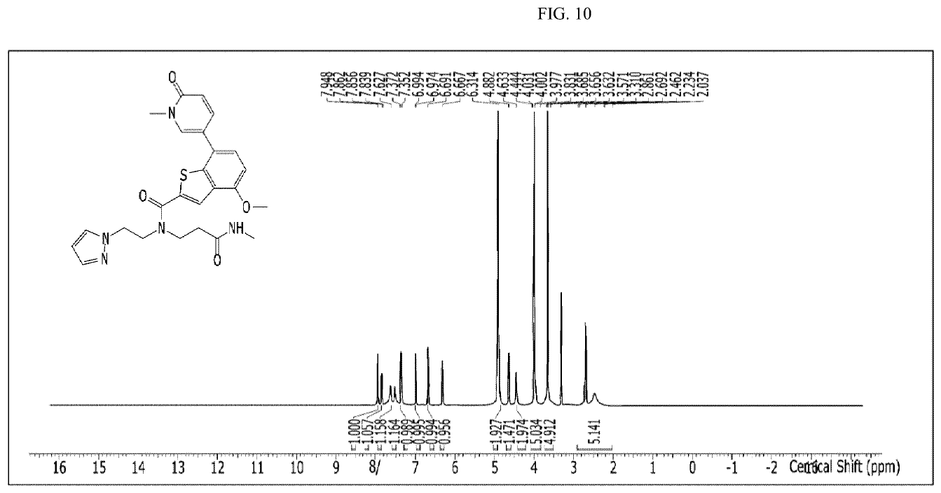

As shown in the synthetic pathway, N(2-(1H-pyrazol-1-yl) ethyl)-4-methoxy-7-(1-methyl-6-oxo-1,6-dihydropyridin-3-yl)-N-(3-(methylamino)-3-oxopropyl) benzo[b]thiophene-2-carboxamide (BBC1131) (8.5 mg, 1000% purity) was obtained from methyl 3-((2-(1H-pyrazol-1-yl)ethyl)amino) propanoate (79.9 mg, 253 μmol) in DMF (2 mL). The structure was confirmed by HNMR and the purity was confirmed by LCMS (FIGS. 9 and 10).

LCMS: EB1363-125-P1A1 (M+H)+, 494.4

H NMR: EB1363-125 P1N1, (400 MHz, MeOD)

δ7.95 (d, 1H, J=2.4 Hz), 7.85 (dd, 1H, J=2.4, 9.3 Hz), 7.63 (br s, 1H), 7.50 (br s, 1H), 7.36 (d, 1H, J=8.0 Hz), 6.98 (d, 1H, J=8.0 Hz), 6.68 (d, 1H, J=9.2 Hz), 6.31 (s, 1H), 4.9-4.9 (m, 1H), 4.9-4.9 (m, 1H), 4.63 (s, 1H), 4.44 (br s, 2H), 3.9-4.1 (m, 5H), 3.6-3.7 (m, 5H), 2.69 (br s, 3H), 2.3-2.6 (m, 2H).

### Example 6: Synthesis of Compound 6 (BBC1680)

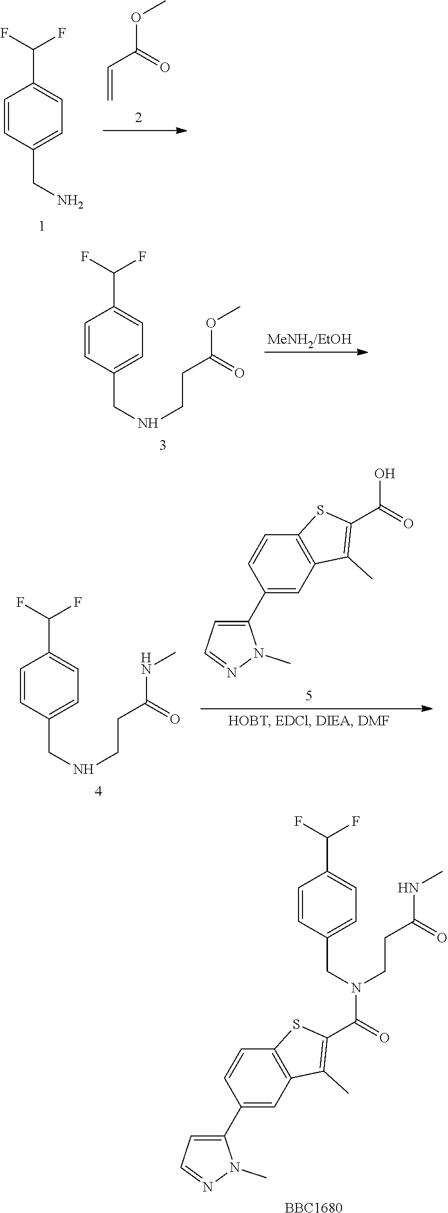

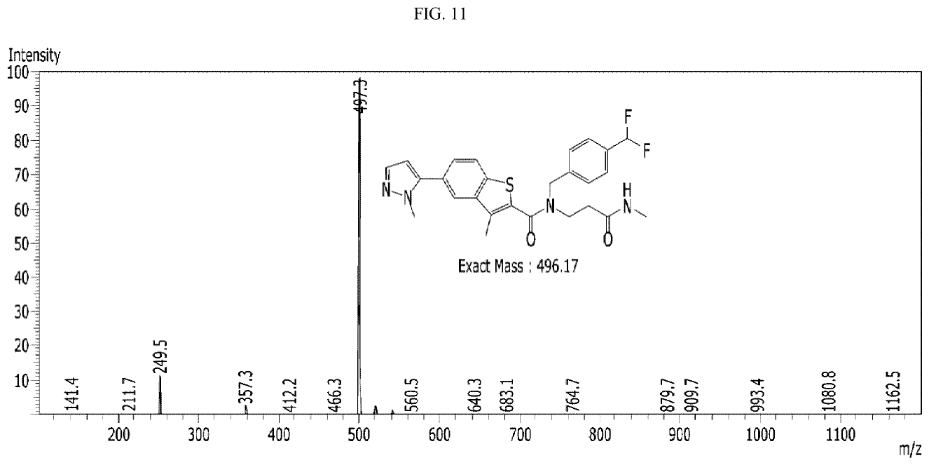

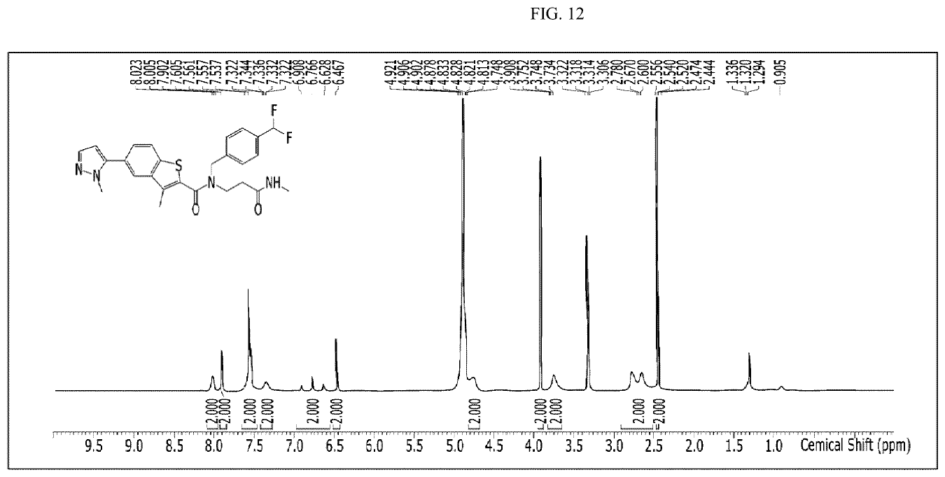

As shown in the synthetic pathway, N-[[4-(difluoromethyl)phenyl]methyl]-3-methyl-N-[3-(methylamino)-3-oxo-propy]-5-(2-methylpyrazol-3-yl)benzothiophene-2-carboxamide (BBC1680) (15.0 mg, 30.1 μmol, 17.4% yield, 99.5% purity) was obtained from [4-(difluoromethyl)phenyl]methanamine (500 mg, 2.58 mmol, 1.0 eq, HCl) in MeOH (5 mL). The structure was confirmed by HNMR and the purity was confirmed by LCMS (FIGS. 11 and 12).

LCMS: EB2777-5-P1A5, m/z=497.3 (M+H)+, Rt=1.617 min.

H NMR: EB2777-5-P1A, 400 MHz, MeOD

δ 8.01 (d, J=7.2 Hz, 1H), 7.90 (s, 1H), 7.47-7.65 (m, 5H), 7.27-7.42 (m, 1H), 6.77 (t, J=56.8 Hz, 1H), 6.46 (s, 1H), 4.68-4.82 (m, 2H), 3.91 (s, 3H), 3.65-3.83 (m, 2H), 2.51-2.91 (m, 5H), 2.44 (s, 3H).

### Example 7: Synthesis of Compound 7 (BBC1674)

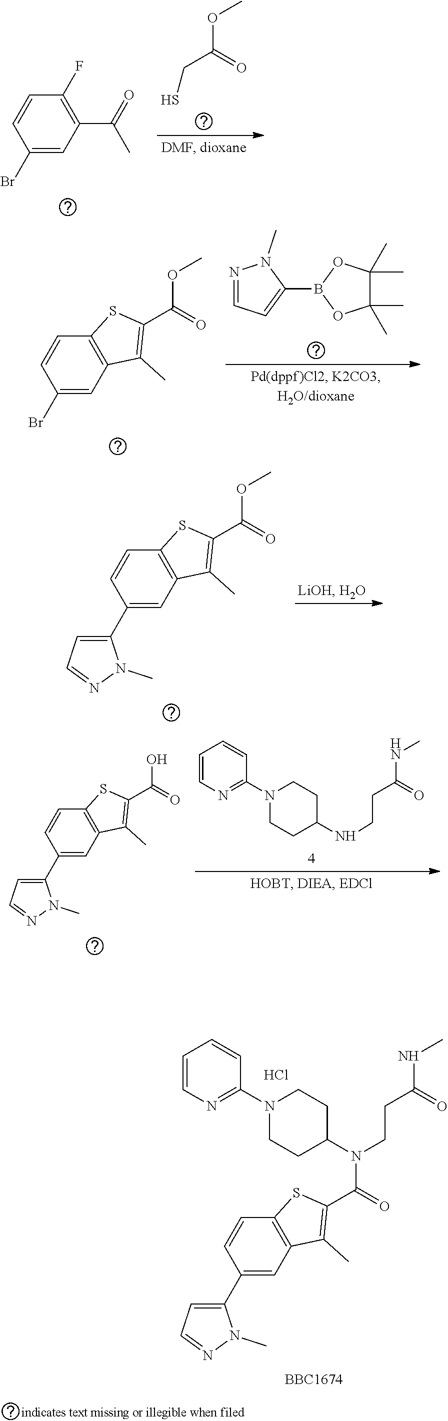

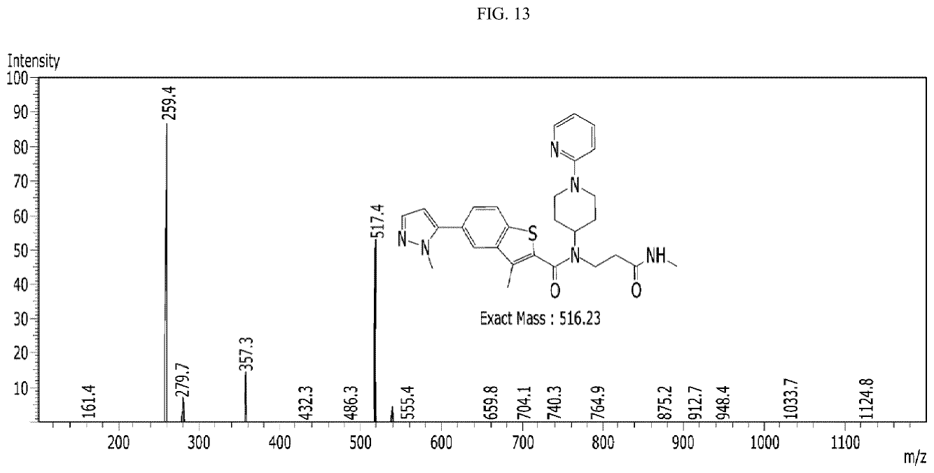

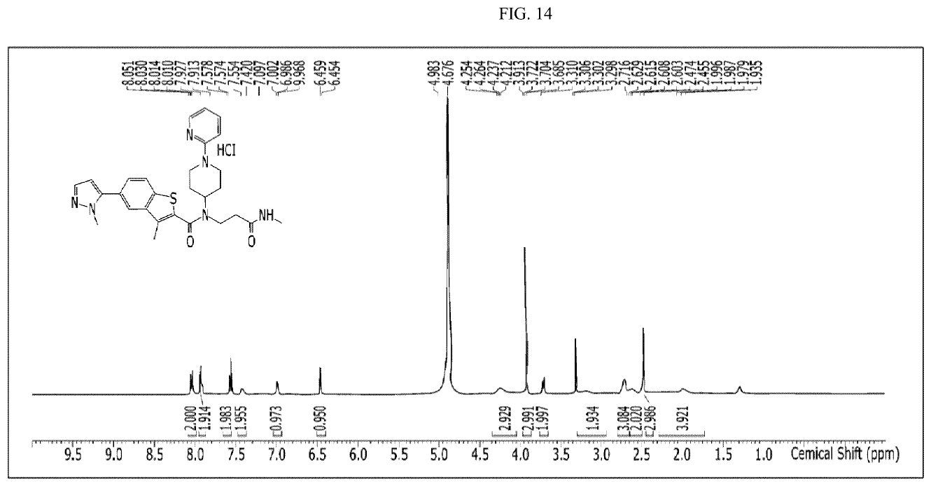

As shown in the synthetic pathway, 3-methyl-5-(1-methyl-1H-pyrazol-5-yl)-N-(3-(methylamino)-3-oxopropyl)-N-(1-(pyridine-2-yl)piperidin-4-yl)benzo[b]thiophene-2-carboxamide (BBC1674) (27.9 mg, 53.0 μmol, 30.7% yield, 98.1% purity) was obtained from methyl 2-sulfanylacetate (978 mg, 9.22 mmol, 836 μL, 2.0 eq). The structure was confirmed by H-NMR and the purity was confirmed by LCMS (FIGS. 13 and 14).

LCMS: EB2777-7-P1B1, m/z=517.4 (M+H)+, Rt=1.460 min.

H NMR: EB2777-7-P1A, 400 MHz, MeOD

δ 7.99-8.10 (m, 2H), 7.89-7.96 (m, 2H), 7.51-7.65 (m, 2H), 7.36-7.48 (m, 1H), 6.99 (t, J=6.4 Hz, 1H), 6.46 (d, J=2.0 Hz, 1H), 4.05-4.35 (m, 3H), 3.91 (s, 3H), 3.70 (t, J=7.2 Hz, 2H), 2.93-3.29 (m, 2H), 2.65-2.83 (m, 3H), 2.50-2.64 (m, 2H), 2.47 (s, 3H), 1.73-2.29 (m, 4H).

### Test Example 1: Binding Inhibitory Test on BRD3 Protein

The binding inhibitory effect of the compounds of the present invention on the BRD3 protein among the BET proteins was tested as follows and confirmed. The compounds were diluted 1:5 serial dilutions in an assay buffer from a 10 mM stock in DMSO in a white OptiPlate. A mixture consisting of 100 nM GST BRD2 (BD1, BD2, BD1+BD2) and 100 nM biotinylated acetyl histone H4 (Lys5, 8, 12, 16) peptides was added to the diluent, and then each sample was incubated with shaking at 300 rpm for 30 minutes at room temperature in the dark. Thereafter, signals were measured with a PerkinElmer Envision HTS Multilabel Reader using a PerkinElmer Alpha Screen protocol. Determination of IC₅₀ values was performed using GraphPad Prism 3.03 software, and the results were shown.

TABLE 2

| Classification | Compound | IC₅₀: BRD3(BD1) |
|---|---|---|
| Example 1 | BBC1115 | 48.95 |
| Comparative example 1 | JQ1 | 261 |
| Comparative example 2 | RVX-208 | 23398 |
| Example 2 | BBC1114 | 35.81 |
| Example 3 | BBC1117 | 61.12 |
| Example 4 | BBC1118 | 29.68 |
| Example 5 | BBC1131 | 51.47 |
| Example 6 | BBC1680 | 98.73 |
| Example 7 | BBC1874 | 84.77 |

As shown in Table 2 above, it was confirmed that the compound of Example 1 of the present invention exhibited excellent BRD protein inhibitory activity, as the IC₅₀ value for BRD3 BD1 was approximately 5.71 times and 532.38 times lower than the IC₅₀ values of Comparative Example 1 and Comparative Example 2, respectively. In addition, it was confirmed that the compounds of Examples 2 to 7 also had significantly lower IC₅₀ values for BRD3 BD1 than Comparative Examples 1 and 2, and had excellent BRD protein inhibitory activity.

### Test Example 2: In Vitro Anticancer Efficacy Test on Human Pancreatic Cancer Cell Line

HPAF, a human pancreatic cancer cell line, was dispensed in a 96-well plate and incubated in a 37° C., 5% CO₂ incubator for 24 hours. Thereafter, 10 μl of Examples and Comparative Examples were treated to each well at different concentrations (1 nM, 0.01 μM, 0.1 μM, 1 μM, 10 μM, 100 μM) and incubated for 96 hours. Each well was treated with a reagent using a CCK assay kit (Dong-In LS, D Plus CCK cell viability assay kit), and reacted in a 37° C., 5% CO₂ incubator for 30 minutes to 1 hour, and then the absorbance was measured at a wavelength of 450 nm using a microplate reader (SpectraMax i3x, USA). The IC₅₀ value was confirmed using the GraphPad Prism 5 (GraphPad Software, Inc., San Diego, USA) program from the results obtained at this time.

TABLE 3

| Classificaton | Compound | IC₅₀ |
|---|---|---|
| Example 1 | BBC1115 | 0.13 uM |
| Comparative example l | JQ1 | 2.3 uM |

As shown in Table 3, it was confirmed that the compound of Example of the present invention exhibited excellent anticancer efficacy, because an IC₅₀ value for the human pancreatic cancer cell line HPAF was about 17.69 times lower than that of Comparative Example 1.

### Test Example 3: In Vitro Anticancer Efficacy Test on AML Cell Line

Changes in MYC protein expression before and after treatment with Examples and Comparative Examples using a mouse-derived AML cell line were confirmed through real-time quantitative PCR (qRT PCR) experiments. Example (BBC1115) and Comparative Example 1 (JQ1) were treated to the cell line at the same concentration (1 μM) for 6 hours, and then RNA was extracted using Trizol. The expression patterns of MYC and HEXIM1 by treatment with test substances were confirmed through qRT PCR.

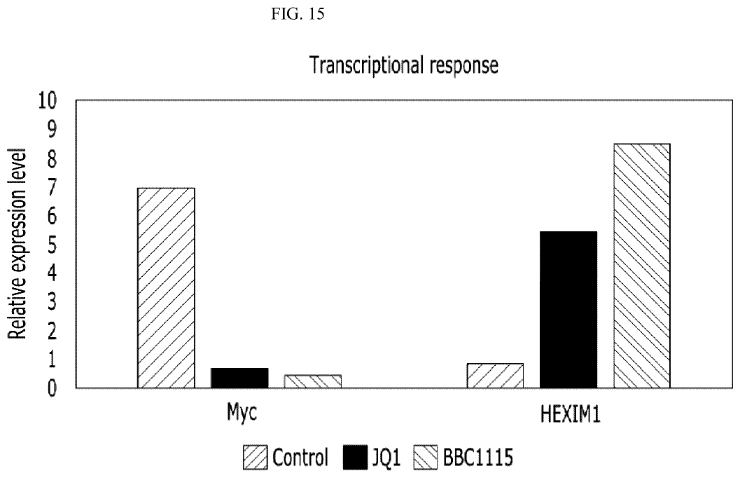

As a result, as shown in FIG. 15, it was confirmed that the compound of Example of the present invention effectively reduced an Myc protein, which was a tumor promoting factor well known as a key target gene of a BET protein, at an mRNA level when treated to a mouse AML cell line under the same conditions as Comparative Example 1. In addition, it was confirmed that the compound of Example was an excellent BET inhibitory anticancer substance by further inducing the expression of the gene HEXIM1, of which expression was known to be increased by a BET inhibitor, compared to Comparative Example.

### Test Example 4: In Vivo Anticancer Efficacy Test

AsPC-1 cells, human pancreatic cancer cells, were placed in a culture flask and incubated in a 37° C., 5% CO₂ incubator. On the day of cell line transplantation, the incubated cells were placed in a tube, centrifuged (1,000 rpm, 5 minutes) to remove the supernatant and make a cell suspension (4×10⁷ cells/mL) with PBS, added with an equal amount of Matrigel, mixed (2×10⁷ cells/mL), and then filled into a disposable syringe, and transplanted by subcutaneously administering 0.2 mL to the right dorsal area of the animal (0.4×10⁷ cells/head). After transplanting the cell line, the tumor volumes were measured for animals with no abnormalities in health conditions after a certain period of time, and animals that reached 100 to 150 mm³ were selected. The test was conducted by dividing into a negative control group, and Example (BBC1115) and Comparative Example (JQ1) administered groups. At this time, Example (BBC1115) was administered at 12, 5, and 25 mg/kg, and Comparative Example (JQ1) was administered at 50 mg/kg, and then the changes in tumor size were observed for 35 days.

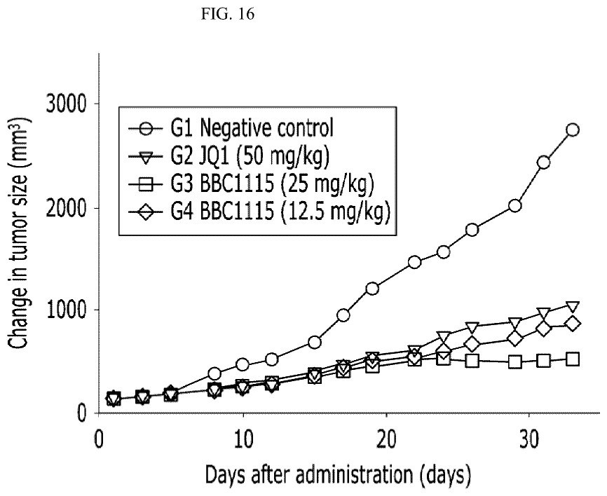

As a result, as shown in FIG. 16, in an anticancer efficacy test using male nude mice transplanted with AsPC-1, a human-derived pancreatic cancer cell line, it was confirmed that the compound of Example of the present invention exhibited significant anticancer efficacy depending on an administered dose and showed superior tumor growth inhibition ability to Comparative Example (JQ1). Therefore, the compound of the present invention has a more excellent inhibitory effect than existing BET inhibitors, and thus can be effectively used for the treatment and prevention of BET-associated diseases.

It will be appreciated by those skilled in the art that the present invention as described above may be implemented into other specific forms without departing from the technical spirit thereof or essential characteristics. Thus, it is to be appreciated that embodiments described above are intended to be illustrative in every sense, and not restrictive. The scope of the present invention is represented by claims to be described below rather than the detailed description, and it is to be interpreted that the meaning and scope of the claims and all the changes or modified forms derived from the equivalents thereof come within the scope of the present invention.

“This study was supported by the Technology Development Project of the Ministry of SMEs and Startups in 2020” [S3029615]

1. A compound represented by Chemical Formula 1 below or a pharmaceutically acceptable salt thereof: 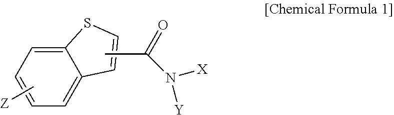
wherein,
X or Y is each independently H, halo, cyano, alkyl, alkenyl, alkynyl, —(Rₐ)C(═O)R_{b}, —(Rₐ)C(═O)N(R_{b})(R_{c}), —(Rₐ)C(═O)OR_{b}, cycloalkyl, heterocycloalkyl, aryl, heteroaryl, pyridine, alkyl pyridine, piperidine, piperidinopyridine, indazole, or, 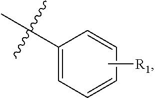
Z is the same or different one or more selected from the group consisting of H, C₁-C₆ alkyl, —ORₐ, —SRₐ, cycloalkyl, heterocycloalkyl, aryl, heteroaryl, pyridine, pyrazole, alkylpyrazole, and hydroxypyridine,
Rₐ, R_{b} and R_{c} are each independently H, alkyl, or alkylene, and
R₁ is H, halo, cyano, alkyl, alkenyl, alkynyl, or halogenated methyl.

2. The compound represented by Chemical Formula 1 or pharmaceutically acceptable salt thereof of claim 1, wherein X is —(Rₐ)C(═O)R_{b} or —(Rₐ)C(═O)N(R_{b})(R_{c}),
Y is 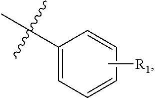
Z is the same or different one or more selected from the group consisting of —ORₐ and hydroxypyridine, and
R₁ is H, halo, cyano, alkyl, alkenyl, alkynyl, or halogenated alkyl.

3. The compound represented by Chemical Formula 1 or pharmaceutically acceptable salt thereof of claim 2, wherein X is —(Rₐ)C(═O)N(R_{b})(R_{c}), and
R₁ is a halogenated alkyl group such as fluoroalkyl, chloroalkyl, or bromoalkyl.

4. The compound represented by Chemical Formula 1 or pharmaceutically acceptable salt thereof of claim 2, wherein Z is the same or different one or more selected from the group consisting of —ORₐ and 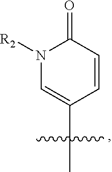
and
R₂ is H or alkyl.

5. The compound or pharmaceutically acceptable salt thereof of claim 1, wherein the compound of Chemical Formula 1 above is selected from the group consisting of the following compounds:

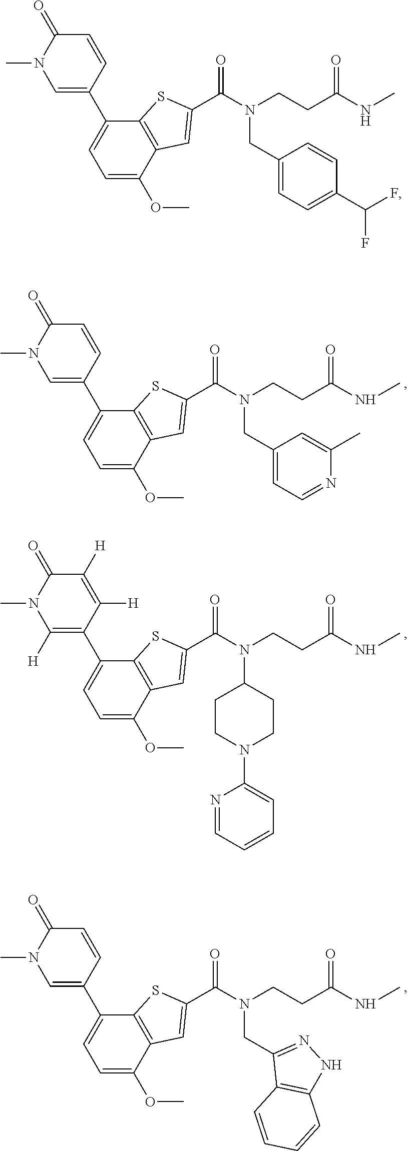

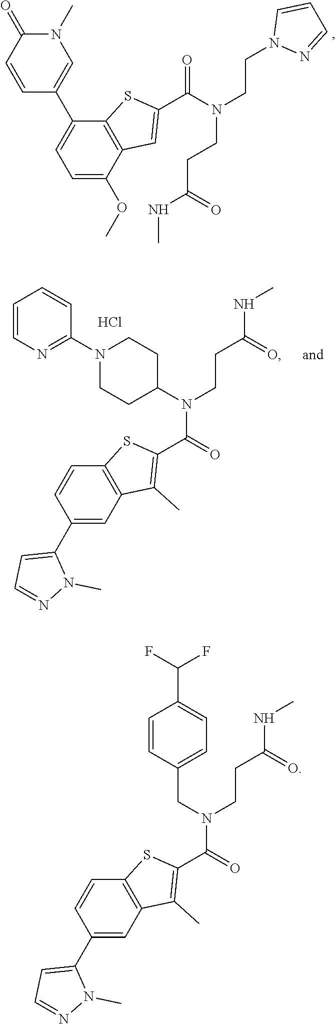 [SMILES: CNC(=O)CCN(CCn1cccn1)C(=O)c1cc2c(OC)ccc(-c3ccc(=O)n(C)c3)c2s1 ; CNC(=O)CCN(C(=O)c1sc2ccc(-c3ccnn3C)cc2c1C)C1CCN(c2ccccn2)CC1 ; Cl ; CNC(=O)CCN(Cc1ccc(C(F)F)cc1)C(=O)c1sc2ccc(-c3ccnn3C)cc2c1C]

6. A pharmaceutical composition for preventing or treating bromodomain extra-terminal (BET) protein-associated diseases, comprising the compound or pharmaceutically acceptable salt thereof according to claim 1.

7. The pharmaceutical composition of claim 6, wherein the BET protein-associated disease is at least one selected from the group consisting of cancer; autoimmune or inflammatory diseases; metabolic diseases; and viral diseases.

8. The pharmaceutical composition of claim 7, wherein the cancer is at least one selected from the group consisting of hematologic cancer, multiple myeloma, acute myeloid leukemia, malignant lymphoma, aplastic anemia, thymus cancer, ovarian cancer, cervical cancer, breast cancer, colorectal cancer, liver cancer, stomach cancer, pancreatic cancer, colon cancer, peritoneal metastatic cancer, skin cancer, bladder cancer, prostate cancer, thyroid cancer, lung cancer, osteosarcoma, fibrous tumor, and brain tumor.

9. The pharmaceutical composition of claim 7, wherein the autoimmune or inflammatory disease is at least one selected from the group consisting of rheumatoid arthritis, systemic lupus erythematosus, multiple sclerosis, Type 1 diabetes, hyperthyroidism, myasthenia, Crohn's disease, ankylosing spondylitis, psoriasis, autoimmune pernicious anemia and Sjogren's syndrome, allergy, allergic rhinitis, arthritis, asthma, chronic obstructive pulmonary disease, degenerative joint disease, dermatitis, organ rejection, eczema, hepatitis, inflammatory bowel disease, sepsis, sepsis syndrome, septic shock, and nonalcoholic steatohepatitis.

10. The pharmaceutical composition of claim 7, wherein the metabolic disease is at least one selected from the group consisting of hypertriglyceridemia, obesity, hyperlipidemia, hyperinsulinemia, hyperglycemia, arteriosclerosis, hypertension, Type 2 diabetes, and insulin resistance disease.

11. The pharmaceutical composition of claim 7, wherein the viral disease is at least one selected from the group consisting of infantile paralysis, acute hemorrhagic conjunctivitis, viral meningitis, hand, foot and mouth disease, hepatitis, myositis, myocarditis, pancreatitis, epidemic myalgia, encephalitis, common cold, herpangina, foot and mouth disease, asthma, bronchiolitis, bronchitis, chronic obstructive pulmonary disease, pneumonia, sinusitis, otitis media, herpes simplex, herpes zoster, stomatitis, and chickenpox.

12. A pharmaceutical composition for preventing or treating bromodomain extra-terminal (BET) protein-associated diseases, comprising the compound or pharmaceutically acceptable salt thereof according to claim 2.

13. A pharmaceutical composition for preventing or treating bromodomain extra-terminal (BET) protein-associated diseases, comprising the compound or pharmaceutically acceptable salt thereof according to claim 3.

14. A pharmaceutical composition for preventing or treating bromodomain extra-terminal (BET) protein-associated diseases, comprising the compound or pharmaceutically acceptable salt thereof according to claim 4.

15. A pharmaceutical composition for preventing or treating bromodomain extra-terminal (BET) protein-associated diseases, comprising the compound or pharmaceutically acceptable salt thereof according to claim 5.

16. A method of preventing or treating bromodomain extra-terminal (BET) protein-associated diseases, comprising administering to a subject in need thereof the compound or pharmaceutically acceptable salt thereof according to claim 1.

17. A method of preventing or treating bromodomain extra-terminal (BET) protein-associated diseases, comprising administering to a subject in need thereof the compound or pharmaceutically acceptable salt thereof according to claim 2.

18. A method of preventing or treating bromodomain extra-terminal (BET) protein-associated diseases, comprising administering to a subject in need thereof the compound or pharmaceutically acceptable salt thereof according to claim 3.

19. A method of preventing or treating bromodomain extra-terminal (BET) protein-associated diseases, comprising administering to a subject in need thereof the compound or pharmaceutically acceptable salt thereof according to claim 4.

20. A method of preventing or treating bromodomain extra-terminal (BET) protein-associated diseases, comprising administering to a subject in need thereof the compound or pharmaceutically acceptable salt thereof according to claim 5.
